(19) United States
(12) Patent Application Publication
(10) Pub. No.: US 2019/0275347 A1
(43) Pub. Date: Sep. 12, 2019

(54) SYSTEM AND METHOD FOR TREATING AILMENTS WITH LIGHT

(71) Applicant: Paul H. Ouyang, San Jose, CA (US)

(72) Inventor: Paul H. Ouyang, San Jose, CA (US)

(21) Appl. No.: 15/916,087

(22) Filed: Mar. 8, 2018

(51) Int. Cl. A61N 5/06

(52) U.S. Cl. CPC A61N 5/0613; A61N 2005/0647; A61N 2005/0663; A61N 2005/0659; A61N 2005/0652

(57) ABSTRACT

An Array Matrix of red and infrared light emitting diodes (LED) are mounted onto to a flexible printed circuit board (FPCB) to produce a compact and wearable Low Level Light Therapy (LLLT) Device. An effective heat releasing method is employed to enable a long period of continuous use of the LLLT device.

### BACKGROUND

### Field of invention

This specification is related to treating ailments, such as pain with light.

### BACKGROUND OF THE INVENTION

In prior approaches complex circuits, designs, and switches are used to build light emitting devices. Such designs are costly, power wasting and low energy efficient.

### SUMMARY OF THE INVENTION

One example of an embodiment of the present Low Level Light Therapy (LLLT) device provides a method and/or system for providing light therapy that is convenient, portable and/or wearable. A benefit of the present LLLT device is to provide a flexible and bendable LLLT device so that the user can physically bend the device to fit according to his or her specific needs, such as wrapping the device around the arm, neck, lower back or wrists. Constructing the device from a flexible/bendable material allows the LLLT device to make close contact to the desired treatment area.

Another benefit of at least one embodiment of the present LLLT device is to make the LLLT device very light weight and mobile. With a portable 5-volt battery, the user can receive treatment and have benefits of the LLLT device while he or she is walking, driving a car, watching television, or sleeping.

Another benefit of at least one embodiment of the present LLLT device is that the LLLT device provides a low-cost, power efficient, flexible and bendable LLLT device that requires no power control circuit, voltage control circuit, or DC/DC voltage conversion circuit (which changes the voltage from one voltage level to another), or on-off switch box to enable LLLT device to operate.

Another benefit of at least one embodiment of the present LLLT device is to provide a low cost LLLT device that operates with any 5 V battery or any USB interface.

One benefit of at least one embodiment of the present LLLT device is to provide an LLLT device that is capable of providing long term periods of continuous use through uniform heat dissipation. In one embodiment, a backside black nylon holder having an adhesive function, and has a front side of sheer transparent fabric. In this specification, the terms “sheer,” “thin,” “transparent,” and “thin and transparent” are used interchangeably and may be substituted one for the other to obtain different embodiments. The black nylon allows for heat absorption and dissipation from the back side of the LLLT device. The sheer transparent layer allows for heated air to escape, and for colder ambient air to cool the heated LED. The heat transfer provided by the sheer transparent provides a comfortable therapeutic heat treatment for the wearer as well.

Another benefit of at least one embodiment of the present LLLT device is to provide an LLLT device are capable of being worn on or attached to any area of the body for light therapy treatment.

Another benefit of at least one embodiment of the present LLLT device is to provide the LLLT device that can be placed inside an object such as a hat to be wearable by a patient. An array matrix of LEDs may be integrated onto a flexible double layered copper printed circuit board (PCB) using surface mount technology (SMT). In an embodiment, an arrangement of the array is includes a copper region that is bendable and an unbending or rigid device region, as will be explained further below. The flexible PCB is placed in a sandwich between a nylon-based bottom layer, and a sheer transparent fabric on top, having the bottom and top (the nylon-based bottom and sheer transparent fabric) sewed together. In addition to this sandwich, self-mounting tape (e.g., double sided tape) can be applied to either front or back side of the LLLT device.

### BRIEF DESCRIPTION OF THE DRAWINGS

FIG. 1A is an embodiment the circuit schematic diagram of an element array of the LLLT device.

FIG. 1B shows the perspective view of the top copper layer interconnection of the basic element array of an embodiment of the LLLT device in conjunction with FIG. 1A.

FIG. 1C shows the perspective view of top copper layer interconnection of the basic building element array of an embodiment the LLLT device with the active devices in place in accordance with FIG. 1A.

FIG. 1D is the symbol of basic element array of the LLLT device of FIG. 1A

FIG. 2A is an embodiment of the LLLT device having a 3 by 12 matrix of basic element array.

FIG. 2B shows the perspective view of layout and dimension of an embodiment of the LLLT device of FIG. 2A.

FIG. 2C shows the structure of the LLLT with the insulator/dielectric layer having the electrodes attached.

FIG. 3A is a perspective view of the construction of an embodiment of the LLLT device.

FIG. 3B is a perspective view of an example of using the LLLT device.

FIGS. 3C and 3D show the increase of temperature with time of an embodiment of the LLLT device.

FIG. 4 depicts an embodiment of the LLLT device using loop fastener.

FIG. 5 depicts an embodiment of the LLLT device with a double side adhesive or mounting tape on the backside.

FIG. 6 depicts an embodiment of the LLLT device with double side adhesive or mounting tape on the front side.

### DETAILED DESCRIPTION OF THE PREFERRED EMBODIMENTS

In the following detailed description of the various embodiment of the LLLT device, numerous specific details are set forth in order to provide a thorough understanding of various embodiment of the LLLT device. However, one or more embodiment of the LLLT device may be practiced without these specific details. In other instances, well-known methods, procedures, and/or components have not been described in detail so as not to unnecessarily obscure aspects of embodiment of the LLLT device.

In the following detailed description of the various embodiment of the LLLT device, reference is made to the accompanying drawings that form a part hereof, and in which is shown by way of illustration a specific embodiment in which the LLLT device may be practiced. It is to be understood that other embodiment may be utilized, and structural changes may be made without departing from the scope of the present LLLT device. Accordingly, the drawings and detailed description are to be regarded as illustrative in nature and not restrictive. In addition, the reference or non-reference to an embodiment of the LLLT device shall not be interpreted to limit the scope of the LLLT device.

Low Level Light Therapy (LLLT) Device using a combination of red and infrared light emitting diodes (LED) to stimulate natural healing function. More particularly, the present LLLT device relates to a wearable LLLT Device using 5V DC battery operation capable of long periods of continuous use.

Low Level Light Therapy (LLLT), also known as photo-bio-modulation, is the application of light energies to biological tissues to stimulate tissue healing and regrowth. Red and infrared lights have wavelengths that are effective in penetrating through the skin and are absorbed by blood vessels and cell tissues. The red and infrared lights has the effect of influencing the localized release of nitric oxide, causing smooth muscles and blood vessels to relax. Light energy also causes certain photo-reactive enzymes to accelerate their functions, thereby enhancing cellular metabolism, circulatory improvement and nerve function, all of which contribute to healing.

A benefit of the present LLLT device is to provide an optimized, low cost design, energy efficient USB based LLLT device. It is particularly the desire of the present LLLT device to have a portable LLLT device, to interface with a portable USB power bank, so that one can wear the LLLT device while carrying the USB battery with him or her.

A benefit of the LLLT device to provide an LLLT device conformable and suitable to be worn on different parts of the body, such as the neck, lower back, knees, elbows, and wrists.

Without the cooling mechanism of the current LLLT device, the device must be turned off after a certain amount of time. Without the cooling mechanism of the current LLLT device both red and (NIR) LED, will experience a premature failure resulting from thermal runaway with long time periods of continuous use. Without the cooling mechanism of the current LLLT device both red and (NIR) LED, 30% of power is converted into light while 70% of power is converted into heat that heats the LLLT device, and the heat generated by the LED device will accumulate. As a result, without the cooling mechanism of the current LLLT device the temperature will continuously rise, and at higher temperature, LED will generate more current and heat, which will in turn make temperature even higher. Without the cooling mechanism of the current LLLT device, when the temperature reaches high enough such as 45-degree C or higher, it can cause discomfort and burning sensation to the skin. These heat issues have not been effectively dealt managed in the past.

In an embodiment, the LLLT device that can effectively disperse the heat to eliminate thermal runaway, so that one can have long time periods of continuous use for maximal healing benefits.

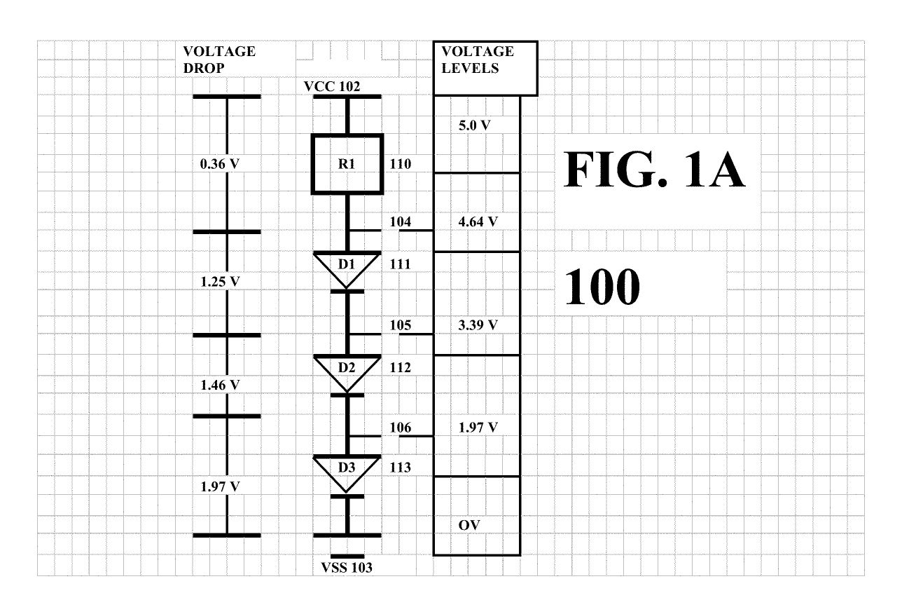

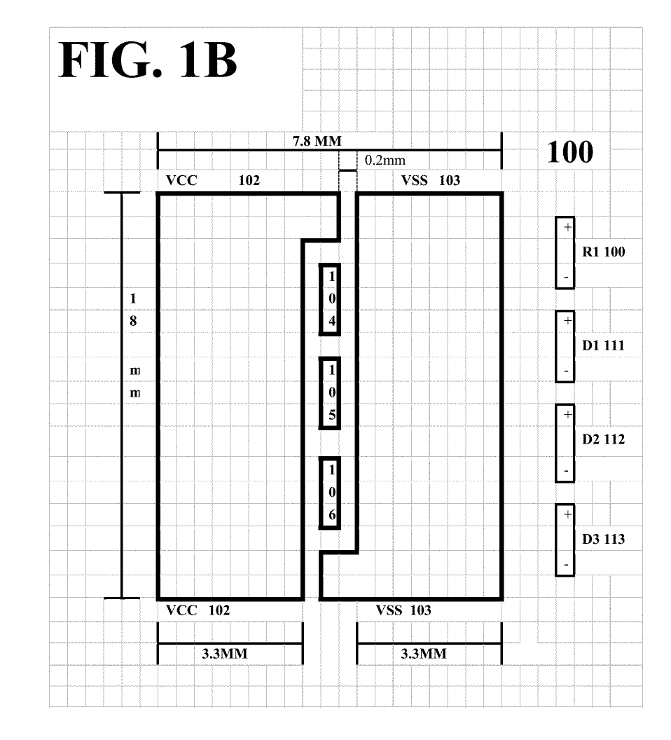

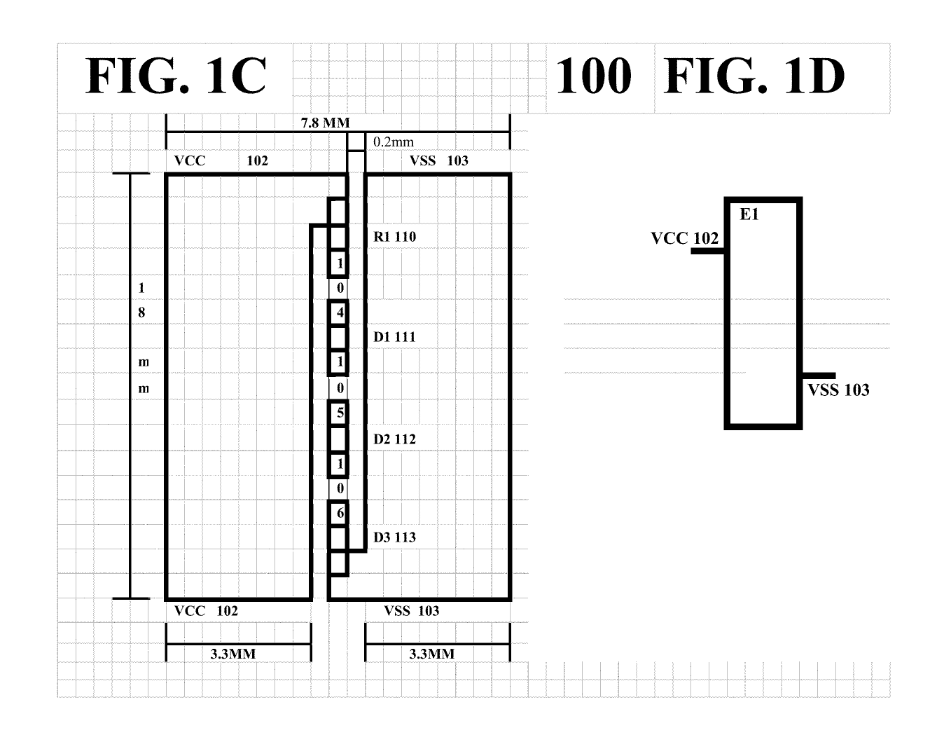

FIG. 1A shows the circuit schematic diagram of a basic element array 100 of the LLLT device of one embodiment of the present LLLT device. The LLLT is includes at least an array of the basic element array which is the only building block used for the LLLT device of the present LLLT device. An embodiment basic element array 100 includes at least a current limiting resistor R1 110, a light emitter D1 111 and a second light emitter diode D2 112, and a third light emitter D3 113 connected in series. Both light emitter D1 111 and D2 112 are NIR LED diodes have a nominal wave length of 850 nm and 940 nm, respectively, as an example of embodiment of the present LLLT device. The term “nominal” refers to the rating that the device is rated at. For example, an LED having a nominal forward resistance of 25 ohms is rated by the manufacturer as having a forward resistance of 25 ohms, but may have a resistance that is slightly different than 25 ohms (e.g., 25 ohms plus/minus 10% of 25 ohms), depending on the quality of the resistor. It is to be noted that Near Infrared (NIR) light is invisible to the naked eye. Light emitter D3 113 is red LED has a wave length of 620 nm. It is to be noted that the arrangement of these diodes is interchangeable.

Resistor R1 110 has a nominal value of 40 ohm in a surface mount package such as type 0603 type. Value of Resistor R1 110 can be in the range of 25 to 150 ohm for most efficient light conversion.

Light emitter D1 111 and D2 112 are near infrared diode having wavelength in the range of 700-1100 nm and forward voltages in the range 0.9-1.5V respectively. Light emitter D3 113 is a red LED, having a nominal wavelength of 550-700 nm and a nominal forward voltage of 1.6-2.2V.

As D1 111 D2 112 and D3 113 are connected in series, and the sum of their forward voltages is 4.5-4.8V, which is less than 5V which is a standard voltage used by any USB device, or interface making such combination ideal to be used with a 5-volt USB battery operation. Thus, no other DC to DC voltage conversion means are needed, nor any an on-off switch means required.

The red LED D3 113 is also used as a quick visual indicator for easy manufacturing purpose. If the red light is turned on when power is applied, indicates that the serial diodes interconnect is okay even though the NIR light cannot be seen with naked eyes. Therefore, having the red LED in the LLLT device, makes testing and repair of the LLLT device in manufacturing easier.

FIG. 1A shows that the positive end of Resistor R1 110 is connected to VCC 102 which is the power source and the negative end of R1 110 is connected to the node 104 which is in turn connected to the positive terminal of D1 111. The negative terminal of D1 111 is connected node 105 which is connected to the positive terminal diode D2 112. The negative terminal of D2 112 is connected to node 106 which is connected to the positive terminal of diode D3 113 whose negative terminal is connected to ground which is VSS node 103

The operation in the basic element array 100 of the LLLT device of the present LLLT device in FIG. 1A is described as follows: VCC 102 is to be connected to an external USB type power supply having a standard 5V with +−5% variation. When 5 V power is applied to the element in FIG. 1A, a current of nominal value of 9 mA will flow through the resistor RI 110, diode D1 111, diode D2 112, diode D3 113 to the VSS ground 103.

The corresponding voltages at nodes 104, 105 and 106, will be 4.640 V, 3.39V, and 1.97V respectively. In other words that the voltage drops across the resistor R1 110, diode D1 111, diode D2 112, diode D3 113, are 0.360V, 1.25V, 1.47V, and 1.97V respectively as shown in FIG. 1A.

Therefore the use of a resistor R1 110 having a resistance of 40 ohm and a 9 ma current flowing through, will result in a 0.36 v or 360 mv drop from VCC, and a 4.640 v drop across the serial diodes D1 111, D2 112 and D3 113 to ground. As Power is the product of current and voltage drop, the current design of this embodiment is very power efficient. The power used or wasted by the resistor is on 7.2% (0.36/5), and the power efficiency of the light emitter diodes are 92.8% (4.64/5). This efficiency is far better than using any DC/DC voltage converter, whose nominal efficiency is less than 80%. Thus, by using 1 red diode and 2 NIR diode and a resistor serially is the most power efficient configuration for a 5 volt operation.

FIG. 1B shows the perspective view of the top copper interconnection layer of the basic element array 100 of the LLLT device in conjunction with FIG. 1A. Both top layer VCC 102 and VSS 103 run in the vertical Y direction. Both VCC and VSS has a nominal width of 3.3 mm. In other embodiments, other widths may be used. A narrow surface mount device R1 110, D1 111, D2 112 and D3 113 are selected and placed along the Y direction, having a width of 0.8 mm (0603 package). In other embodiments, other widths may be used. However, other packages with different width can also be used. Node 104, node 105, and node 106 are top layer metal interconnects for node 114, 115, 116 respectively.

The width of the base element array 100 of FIG. 1A is 7.8 mm in the present example having a metal to metal spacing of 0.2 mm for ease of manufacturing. In other embodiments, other widths may be used. It is to be noted that only bending in the Y-direction is allowed. The bendable area is in the direction of the VCC and VSS lines. The bendable width is 6.6 mm of a total width of 7.8 mm, which is 85%. In other embodiments, other bendable widths may be used. The unbendable area is the device area of 0.8 mm width in the present example. In other embodiments, other widths may be used. The length of the basic element array 100 is chosen to be 18 mm as shown in FIG. 1B in our present example. In other embodiments, other widths may be used.

It is to be noted that there are no resistors or diodes that are to be placed in the bending area along top VCC and VSS tracks to ensure easy bending.

It is also to be noted that the wide width of VCC 102 and VSS 103 lines are highly heat conductive and are beneficial to distribute heat uniformly when power is applied to remove isolated hot spot.

FIG. 1C shows the placement of passive devices R1 110, D1 111, D2 112 and D3 113 placed in between the top VCC 102 and VSS 103 copper layer making them electrically connected as is in FIG. 1A. In this specification, any place copper is mentioned other conductors may be substituted, such as aluminum, silver, gold, iron, lead, platinum, for example.

FIG. 1D is a symbol of the base element array 100 of the circuit of FIG. 1A. It has 2 terminals VCC 102 and VSS 103.

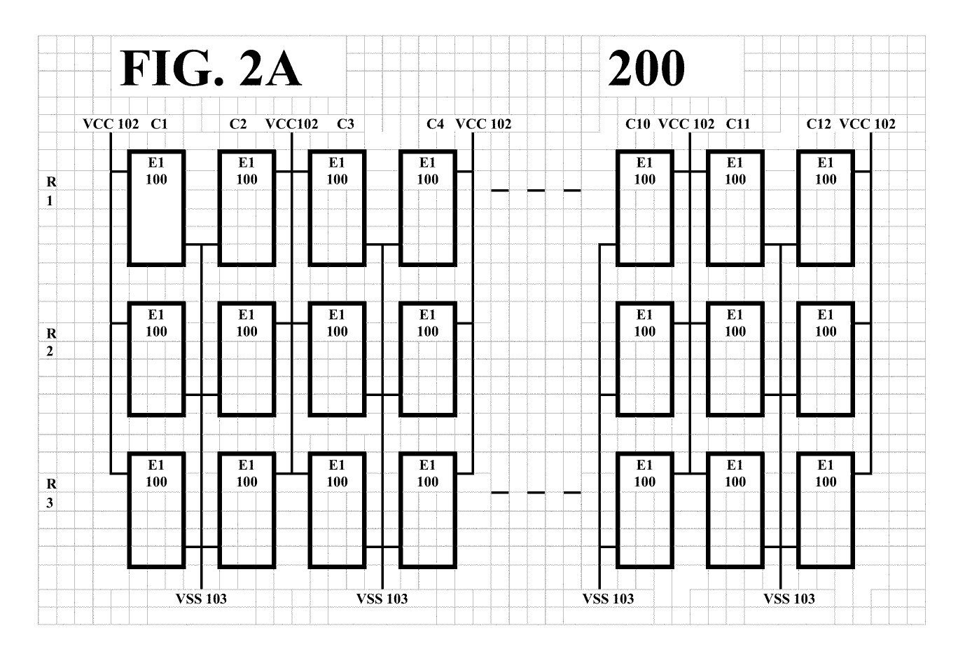

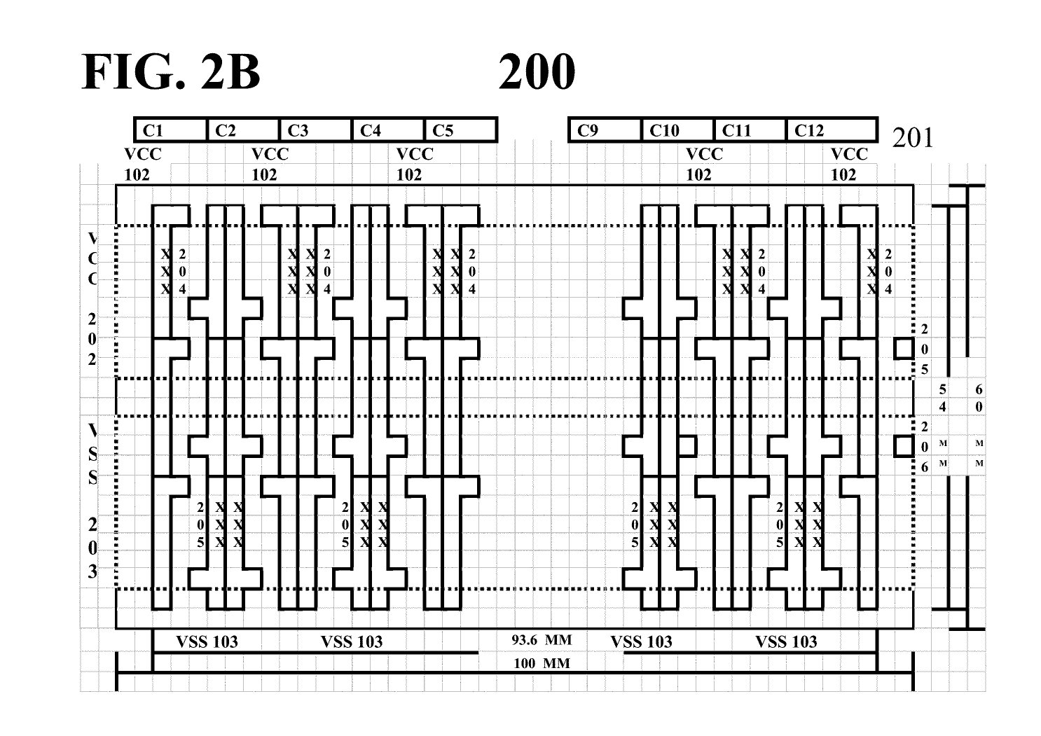

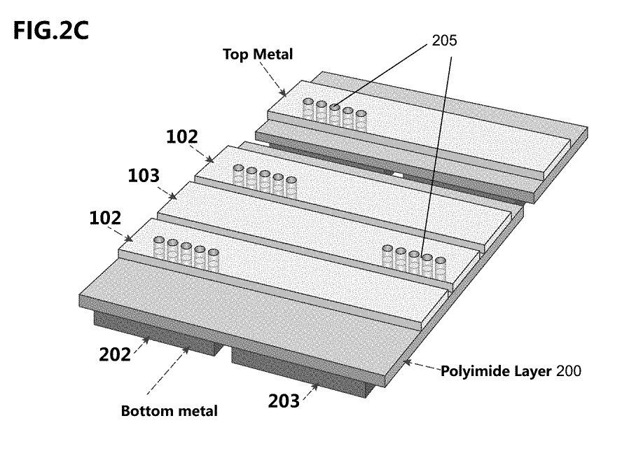

FIG. 2A shows the schematic of a flexible light circuit device 200 of an embodiment of present LLLT device. The flexible circuit device 200 includes at least a 3×12 matrix of the basic element array 100 of FIG. 1A, having 3 rows and 12 columns of the basic element array 100 of an embodiment of the LLLT device arranged in parallel electrically. As each column has 3 basic element arrays 100, and each basic element array 100 has of 3 diodes, there are 9 diodes is placed in each column. With 12 columns, the flexible light circuit device 200, has a total of 108 LED diodes. The dimensions of each basic element array may be 7.8 mm×18 mm, the dimension of the matrix of the basic element array is therefore 93.6 mm×54 mm placed on a 100 mm×60 mm flexible PCB layer 201 as shown in FIG. 2B. In other embodiments, other widths may be used.

When a 5 volt is applied to the flexible light circuit device 200, each basic element array will dissipate 9 ma. The total power dissipation from the flexible light circuit device 200 will be 324 ma (9 ma×36). The total power dissipation is therefore 1.62 Watt (5×0.324 Watt), or 27 mW/cm² (1.62 W/60 cm²).

FIG. 2B depicts the layout of the flexible circuit device 200 of an embodiment of the present LLLT device of FIG. 2A. Each column in FIG. 2B has VCC 102 and VSS 103 runs in the vertical (Y) direction. Column C1 and Column C2 are mirrored in the Y direction. So that they can share the same VSS 102. The combination of C1 and C2 are then repeated, to column c3, c4; 5, c6; c7, c8; c9, c10; c11 and c12.

FIG. 2B also shows a second copper layer. Said second copper layer is a bottom copper layer comprised of VCC 202, and VSS 203 layer. It is to be noted that there is a dielectric layer or 200 of polyimide separating the bottom copper layer VCC 202 and VSS 203 and top copper layers (not shown). In other embodiments, other insulators may be used instead of polyimide. In this specification the term dielectric and insulator are used interchangeably, either term may be substituted for the other anywhere in the specification to obtain different embodiments. The bottom copper layer VCC 202 is electrically connected to all top VCC copper layer 102 through conductive material (or wires) in holes 204 in the insulator layer 200. Bottom copper layer VSS 203 is connected to all upper VSS layer 103 through via holes 205. The width of the bottom copper layer VCC 202 and VSS 203 are 23 mm respectively. The length of the bottom copper layer VCC 202 and VSS 203 is 100 mm. In other embodiments, other widths may be used. FIG. 2B. also shows that the bottom VCC 202 and VSS 203 are also connected to openings 206 and 207 respectively which are to be connected to the power and ground of an external USB port via soldering. Both Bottom VCC 202 and VSS 203 has a width of 23 mm. The wide width of VCC 202 and VSS 203 lines also distribute heat across the FPCB 201 effectively.

FIG. 2C shows the bending of the flexible PCB in along the Y axis. FIG. 2C shows the structure of the LLLT with the insulator/dielectric layer having the electrodes attached.

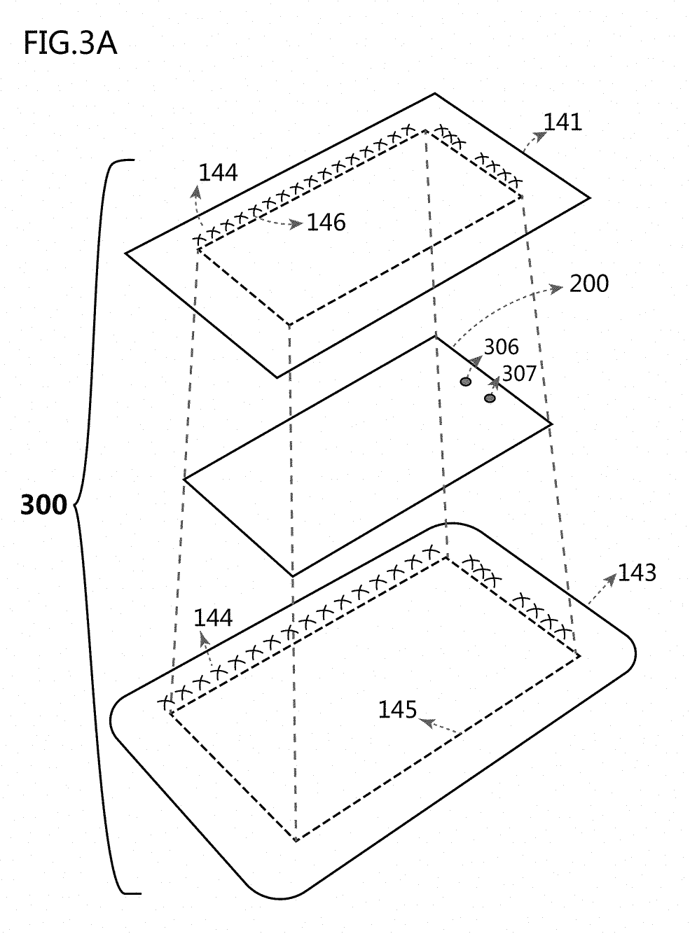

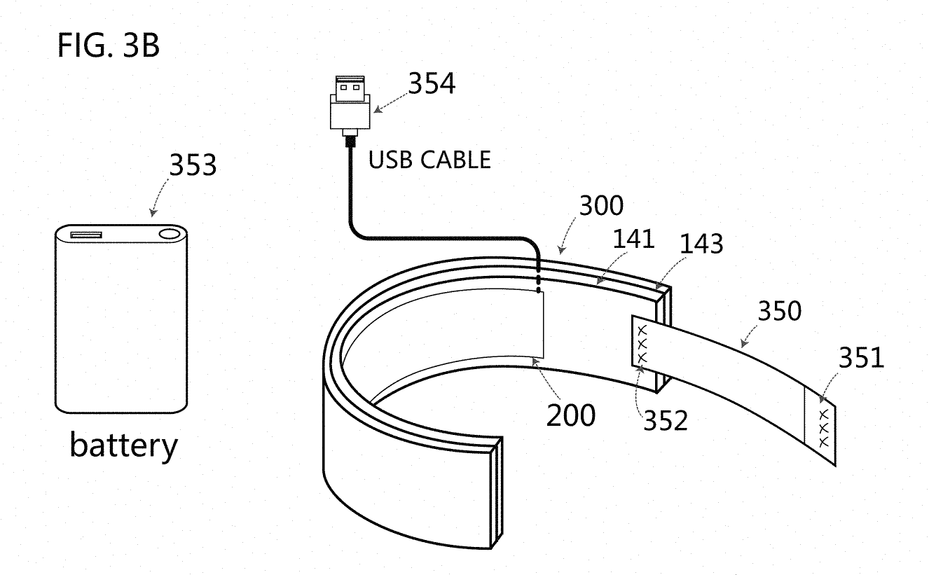

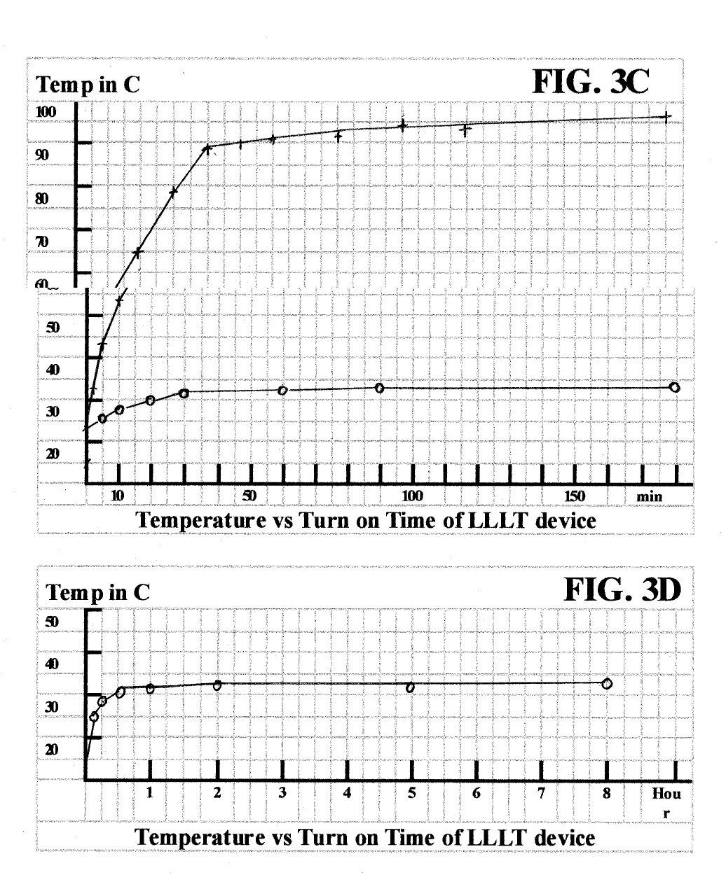

FIG. 3A depicts the LLLT device 300 with housing. Device 300 is comprised of a top layer 141 of a transparent sheer fabric, a flexible circuit device 200 of FIG. 2C and a bottom nylon layer 143. The top sheer transparent fabric is made of nylon threads which is net like with openings. It allows most of the light from the light circuit device 200 to penetrate through directly or scatter through to the treatment area of the user. This fabric also allows air exchange freely.

The heat generated by the light emitting diodes will cause temperature to rise. At higher temperature, the heated air will quickly expand and escape to the surrounding area through top layer 141 and be replaced by the cooler surrounding air. This cold air will reduce the junction temperature of the light emitting diode. This heat dispersion mechanism is important for the LLLT device to be used continuously for a long period of time without further rising of temperatures.

The middle layer 200 is the flexible device where arrays of the basic element of the present LLLT device are placed. Nodes 306 and 307 are nodes that correspond to nodes 206 and 207 of FIG. 2B. Nodes 306 and 307 are the power connections of the VCC and VSS terminals to the USB terminal.

The layer 143 is a dark nylon material, which can absorb and transmit heat from the backside of the FPCB layer 201 of FIG. 2B of the current LLLT device. Layers 141 and 143 are fastened (e.g., sewn) together. The flexible light emitting device 200 is secured as a sandwiched between layers 141 and 143. Heat generated by the array of LED and resistors will distribute uniformly through both sides of the FPCB 201, as VCC 202/VSS 203 on the bottom and VCC 102/VSS 103 on the top are good heat conducting layers. Heat from the Flexible light emitting device 201 are dispersed to the surrounding both through hot air exchange on the front side, and back side black body absorption and radiation. When heat generation and heat dispersion come into equilibrium, temperature and current used by the LLLT device will be constant. Thus, the LLLT device of the present LLLT device can allow long periods of continuous use.

It is to be noted that layer 141 is larger than device 200; layer 143 is equal to or larger than layer 141. FIG. 3A shows the placement mark 145 and 146 of the light emitting device 200 on the bottom and top layer 143 and 141 respectively, and the sawing marks 144, which will secure the device 200. The sawing marks 144 is sown around the placement mark 145 and 146. However, it is only partially shown for clarity purposes.

FIG. 3B Shows the LLLT device 300 to be connected to the battery 153 through a USB cord 154. The LLLT device 300 is connected to a strap 150 and hook 151. The strap 150 can wrap around the user's wrist, arm, neck or knee. It is to be noted that both layer 143 and strap 150 are made of nylon material with loop.

FIGS. 3C and 3D show the increase of temperature with time of an embodiment of the LLLT device. FIG. 3C shows a comparison of the temperature verses time with and without the cooling mechanism described herein. The lower curve having the cooling mechanism and the higher curve does not have the cooling mechanism. As one can see with the cooling mechanism, the temperature levels off to less than 40 degrees centigrade in less than an hour and remains at less than 40 degrees centigrade indefinitely. Whereas without the cooling mechanism, the temperature climbs to close to 100 degrees centigrade within about 30 to 40 minutes (which becomes intolerable for most people) and continues to climb towards 100 degrees centigrade for several hours.

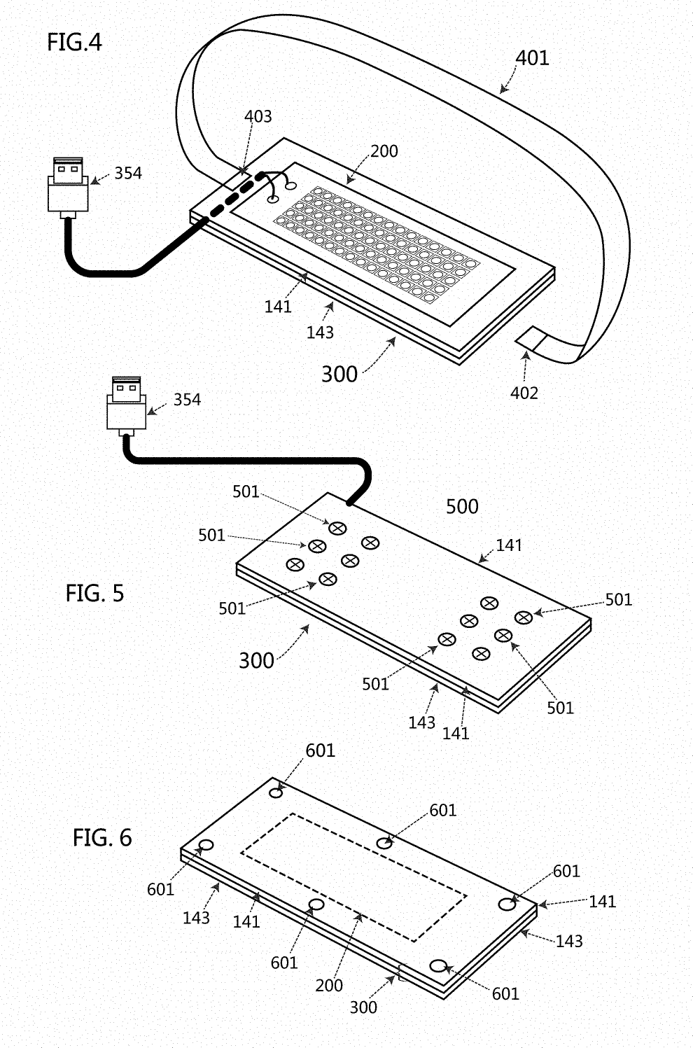

FIG. 4 is an embodiment of wearable LLLT device 300, which is connected to a loop fastener 401. The loop fastener 401 is also made of nylon loop, which is same as of layer 143. Both ends of the loop fastener 401 are sowed with hooks 402 and 403. By using different length for the loop fastener 401, one can easily wrap around different part of the body such as wrists, arms, knees, neck, chest, or lower back.

FIG. 5 depicts a LLLT device 500 with double sided mounting tape 501 on layer 143 of the LLLT device 300 or the backside of the LED emitters. The LLLT device 500 can be placed and secured through the mounting tape 501 inside of a hat (not shown), or the inside of a wearable piece of clothing to target a potential treatment area. The LLLT device 500 can also be placed and secured on a chest belt, or a wrist wrap.

FIG. 6 depicts a wearable LLLT device 600, with the double sided sticky adhesive bands 601 placed on the same side of LED emitters. This wearable LLLT device 600 can then be directly adhered to the treatment area of the body with the adhesive 601 holding the device in place. Optionally, Velcro®, another hook and loop material, or another fastener (buckles, zippers, buttons, or clasps, for example) may be used to close band 601 (of different embodiments of the LLLT device) on a user's wrist arm, hand, finger, leg, foot, toe, torso, head, neck, or other body part.

In any of the embodiments of the wearable LLLT device, the flexible printed circuit board may comprise an insulative layer (the term “insulative layer” being a layer that insulates); a first conductive layer connected to a ground connector; and a second conductive layer connected to a power connector, the first conductive later and the second conductive layer being separated from one another, and both the first conductive later and the second conductive layer being on one side of the insulative layer.

In any of the embodiments of the wearable LLLT device, the first conductive layer may have a first area, the second conductive layer may have a second area, and the insulative layer may have a third area, such that a sum of the first area and the second area together being greater than half of the third area.

In any of the embodiments of the wearable LLLT device, the flexible printed circuit board may comprise a first plurality of conductive layer electrically connected to the first conductive layer; a second plurality of conductive layer electrically connected to the second conductive layer; each of the first plurality of conductive layers connected to one end of one of a plurality of heating elements; and each of the second plurality of conductive layers connected to a second end of one of the plurality of heating elements.

In any of the embodiments of the wearable LLLT device, the first plurality of conductive layers and the second plurality of conductive layers may be located on a second side of the insulative layer that is opposite the side of the insulative layer having the first conductive layer and the second conductive layer.

In any of the embodiments of the wearable LLLT device, the first plurality of conductive layers may have a fourth area and the second plurality of conductive layers may have a first area, the fourth area and fifth area summing to more than a third of the third area.

In any of the embodiments of the wearable LLLT device, the first plurality of conductive layers and the second plurality of conductive layers may be perpendicular to the first conductive layer and the second conductive layer.

ALTERNATIVES AND EXTENSIONS

Each embodiment disclosed herein may be used or otherwise combined with any of the other embodiments disclosed. Any element of any embodiment may be used in any embodiment.

Although the invention has been described with reference to specific embodiments, it will be understood by those skilled in the art that various changes may be made and equivalents may be substituted for elements thereof without departing from the true spirit and scope of the invention. In addition, modifications may be made without departing from the essential teachings of the invention.

What we claim:

1. A wearable Low Level Light Emitting Diode Therapy (LLLT) device comprising:
a flexible circuit board having a circuit attached; and
USB power connection;
wherein said flexible circuit device is includes at least a flexible printed circuit board, a matrix of element arrays, a power supply connecting node and a ground connecting node;
wherein said flexible printed circuit board (PCB) has a first and a second conductive layer;
wherein said first conductive layer has a plurality of power and ground lines running in one direction,
wherein said second conductive layer has a power line and a ground line running perpendicular to said first conductive layer;
wherein the ground line of said second conductive line is connected to all the ground lines of said first conductive layer via holes;
wherein the power line of said second conductive line is connected to all power lines of said first conductive layer via holes;
wherein the element array includes at least a resistor, a first near-infrared (NIR) diode, a second NIR diode and a third red LED diode connected in series;
wherein said resistor, said first NIR diode, said second NIR diode and said red LED diode are placed between the said power and ground line of the first said conductive layer;

2. The wearable LLLT device of claim 1 is bendable along the direction of the said first conductive layer of the flexible PCB.

3. The wearable LLLT device of claim 1 wherein the sum of the forward voltage of the first NIR diode, the second NIR diode and the third red LED diode are less than 5 volts.

4. The wearable LLLT device for personal use of claim 1 wherein the value of said resistor is in the range of 25-100 ohm.

5. The wearable LLLT devise of claim 1, further comprising:
a top layer;
a bottom supporting layer;
wherein said top layer and said bottom layer are fastened together with the flexible circuit substrate fixed in between.

6. The wearable LLLT device of claim 5, said top layer allows free air exchange with the surroundings.

7. The wearable LLLT device of claim 5, said top layer is made of nylon fabric.

8. The wearable LLLT device of claim 5, said bottom support layer is thermally conductive.

9. The wearable LLLT device of claim 5, said bottom support layer is nylon material or thermally conductive nylon silicone.

10. The wearable LLLT device of claim 5, further comprising
a body strap;
wherein said body strap is attached to said top layer and holds said LLLT device around anybody part.

11. The wearable LLLT device of claim 5, further comprising
double sided tap tapes.

12. The wearable LLLT device of claim 9,
wherein double sided tape is attached to said bottom layer.

13. The wearable LLLT device of claim 10,
wherein said bottom layer is further connected to an external wearable device.

14. The wearable LLLT device of claim 11,
wherein said bottom layer is further connected to a cap for head wear.

15. The wearable LLLT device of claim 9,
wherein self-mounting tape is attached to said top layer.

16. The wearable LLLT device of claim 13,
wherein self-mounting tape is attached to said top layer.

17. A method comprising: attaching the wearable LLLT device of claim 13 to a treatment area.

18. A wearable Low Level Light Emitting Diode Therapy (LLLT) device comprising:
a flexible circuit board having a heating circuit attached;
the flexible circuit board being sandwiched between a flexible heat absorbing layer; and
a mesh material that allows the heat to escape.

19. The wearable LLLT device of claim 18, the heating circuit including at least an array of heating elements.

20. The wearable LLLT device of claim 19, each heating element including a series of near infrared (NIR) light emitting diodes.

21. The wearable LLLT device of claim 20, further including a light emitting diode of another wavelength chosen that a combination of the series of NIR diodes and the diode of another wave length have an efficiency of more than 95% for a given voltage source.

22. The wearable LLLT device of claim 21, the diode of another wave length being a red light emitting diode.

23. The wearable LLLT device of claim 22, the voltage source being a Universal Serial Bus (USB) power connection.
